(19) United States
(12) Patent Application Publication
(10) Pub. No.: US 2010/0130756 A1
(43) Pub. Date: May 27, 2010

(54) METHOD FOR PRODUCING TRIOXANE FROM TRIOXYMETHYLENE GLYCOL DIMETHYL ETHER

(75) Inventors: Eckhard Stroefer, Mannheim (DE); Neven Lang, Mannheim (DE); Markus Siegert, Heidelberg (DE); Hans Hasse, Kaiserslautern (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/593,964

(22) PCT Filed: Mar. 27, 2008
(86) PCT No.: PCT/EP08/53668

§ 371 Date: Sep. 30, 2009

(30) Foreign Application Priority Data
Mar. 30, 2007 (EP) 07105347.4

(51) Int. Cl. C07D 323/06

(52) U.S. Cl. 549/368

(57) ABSTRACT

A process for preparing trioxane from trioxymethylene glycol dimethyl ether (POMDMEₙ₌₃) by converting trioxymethylene glycol dimethyl ether in the presence of an acidic catalyst and subsequent distillative workup of the reaction mixture, comprising the steps of:
a) feeding trioxymethylene glycol dimethyl ether (POMDMEₙ₌₃) or a mixture comprising trioxymethylene glycol dimethyl ether into a reactor and converting it in the presence of an acidic catalyst to a mixture a comprising trioxane, formaldehyde, water, methylene glycol (MG), polyoxymethylene glycols (MG_{n>1}), methanol, hemiformals (HF), methylal (POMDMEₙ₌₁) and polyoxymethylene glycol dimethyl ethers (POMDME_{n>1});
b) distillatively separating the reaction mixture a into a low boiler fraction b1 comprising trioxane, formaldehyde, water, methylene glycol, methanol, hemiformal (HFₙ₌₁), methylal and dioxymethylene glycol dimethyl ether (POMDMEₙ₌₂), and a high boiler fraction b2 comprising polyoxymethylene glycols (MG_{n>1}), hemiformals (HF_{n>1}) and polyoxymethylene glycol dimethyl ethers (POMDME_{n>2});
c) distillatively separating the low boiler fraction b1 into a low boiler fraction c1 comprising formaldehyde, water, methylene glycol, methanol, hemiformal (HFₙ₌₁), methylal and dioxymethylene glycol dimethyl ether (POMDMEₙ₌₂), and a high boiler fraction c2 comprising trioxane.

Trioxane is generally prepared by distilling aqueous formaldehyde solution in the presence of acidic catalysts. The trioxane is subsequently removed from the distillate comprising formaldehyde and water by extraction with halogenated hydrocarbons, such as methylene chloride or 1,2-dichloroethane, or other water-immiscible solvents.

DE-A 1 668 867 describes a process for removing trioxane from mixtures comprising water, formaldehyde and trioxane by extraction with an organic solvent. In this process, an extraction zone consisting of two subzones is charged at one end with a customary organic, virtually water-immiscible extractant for trioxane, and at the other end with water. Between the two subzones, the distillate of the trioxane synthesis to be separated is fed. On the side of the solvent feed, an aqueous formaldehyde solution is then obtained, and on the side of the water feed, a virtually formaldehyde-free solution of trioxane in the solvent. In one example, the distillate which is obtained in the trioxane synthesis and is composed of 40% by weight of water, 35% by weight of trioxane and 25% by weight of formaldehyde is metered into the middle section of a pulsation column, and methylene chloride is fed at the upper end of the column and water at the lower end of the column. This affords an about 25% by weight solution of trioxane in methylene chloride at the lower end of the column and an about 30% by weight aqueous formaldehyde solution at the upper end of the column.

A disadvantage of this procedure is the occurrence of extractant which has to be purified. Some of the extractants used are hazardous substances (T or T⁺ substances in the context of the German Hazardous Substances Directive), whose handling entails special precautions.

DE-A 197 32 291 describes a process for removing trioxane from an aqueous mixture which consists substantially of trioxane, water and formaldehyde, by removing trioxane from the mixture by pervaporation and separating the trioxane-enriched permeate by rectification into trioxane and an azeotropic mixture of trioxane, water and formaldehyde. In the example, an aqueous mixture consisting of 40% by weight of trioxane, 40% by weight of water and 20% by weight of formaldehyde is separated in a first distillation column under atmospheric pressure into a water/formaldehyde mixture and into an azeotropic trioxane/water/formaldehyde mixture. The azeotropic mixture is passed into a pervaporation unit which contains a membrane composed of polydimethylsiloxane with a hydrophobic zeolite. The trioxane-enriched mixture is separated in a second distillation column under atmospheric pressure into trioxane and, in turn, into an azeotropic mixture of trioxane, water and formaldehyde. This azeotropic mixture is recycled upstream of the pervaporation stage. A disadvantage of this procedure is the very high capital costs for the pervaporation unit.

DE-A 103 61 518 describes a process for preparing trioxane from an aqueous formaldehyde solution, in which an input stream comprising formaldehyde, trioxane and water is prepared in a preceding trioxane synthesis stage from an aqueous formaldehyde solution, and then trioxane is removed from this stream. Alternatively, the trioxane synthesis and the first distillation stage of the trioxane removal can be combined in a reactive distillation.

To this end, in the trioxane synthesis stage, the stream of aqueous formaldehyde solution is converted in the presence of acidic homogeneous or heterogeneous catalysts, such as ion exchange resins, zeolites, sulfuric acid and p-toluenesulfonic acid, at a temperature of generally from 70 to 130° C. It is possible to work in a distillation column or an evaporator (reactive evaporator). The product mixture of trioxane/formaldehyde and water is then obtained as a vaporous vapor draw stream of the evaporator or as a top draw stream at the top of the column. The trioxane synthesis stage can also be performed in a fixed bed reactor or fluidized bed reactor over a heterogeneous catalyst, for example an ion exchange resin or zeolite.

In a further embodiment of the process described in DE-A 103 61 518, the trioxane synthesis stage and the first distillation stage are performed as a reactive distillation in a reaction column. In the stripping section, this may comprise a fixed catalyst bed of a heterogeneous acidic catalyst. Alternatively, the reactive distillation can also be performed in the presence of a homogeneous catalyst, in which case the acidic catalyst is present together with the aqueous formaldehyde solution in the column bottom.

What is common to all processes described in the prior art is that trioxane is prepared under acidic catalysis from aqueous formaldehyde solutions. What is found to be problematic is that trioxane, formaldehyde and water form a ternary azeotrope which, at a pressure of 1 bar, has the composition of 69.5% by weight of trioxane, 5.4% by weight of formaldehyde and 25.1% by weight of water. The removal of pure trioxane from the product mixture of the trioxane synthesis which comprises formaldehyde and water is therefore difficult. According to DE-A 103 61 518 this azeotrope is bypassed by pressure swing distillation, in which a first and a second distillation are performed at different pressures. In a first distillation column which is operated at lower pressure, the starting mixture is separated into a trioxane/water mixture with a low formaldehyde content and an essentially trioxane-free formaldehyde/water mixture. The trioxane-free formaldehyde/water mixture can be recycled into the trioxane synthesis. In a second distillation column operated at higher pressure, the trioxane/formaldehyde/water mixture is separated into pure trioxane and a trioxane/formaldehyde/water mixture with a relatively low trioxane content.

It is an object of the invention to provide a further advantageous process for preparing trioxane. It is a particular object of the invention to provide an advantageous process for preparing trioxane, in which no formaldehyde/trioxane/water azeotropes which are difficult to separate are formed.

The object is achieved by a process for preparing trioxane from trioxymethylene glycol dimethyl ether (POMDMEₙ₌₃) by converting trioxymethylene glycol dimethyl ether in the presence of an acidic catalyst and subsequent distillative workup of the reaction mixture, comprising the steps of:
a) feeding trioxymethylene glycol dimethyl ether (POMDMEₙ₌₃) or a mixture comprising trioxymethylene glycol dimethyl ether into a reactor and converting it in the presence of an acidic catalyst to a mixture a comprising trioxane, formaldehyde, water, methylene glycol (MG), polyoxymethylene glycols (MG_{n>1}), methanol, hemiformals (HF), methylal (POMDMEₙ₌₁) and polyoxymethylene glycol dimethyl ethers (POMDME_{n>1});
b) distillatively separating the reaction mixture a into a low boiler fraction b1 comprising trioxane, formaldehyde, water, methylene glycol, methanol, hemiformal (HFₙ₌₁), methylal and dioxymethylene glycol dimethyl ether (POMDMEₙ₌₂), and a high boiler fraction b2 comprising polyoxymethylene glycols (MG_{n>1}), hemiformals (HF_{n>1}) and polyoxymethylene glycol dimethyl ethers (POMDME_{n>2});
c) distillatively separating the low boiler fraction b1 into a low boiler fraction c1 comprising formaldehyde, water, methylene glycol, methanol, hemiformal (HFₙ₌₁), methylal and dioxymethylene glycol dimethyl ether (POMDMEₙ₌₂), and a high boiler fraction c2 comprising trioxane.

In step a), trioxymethylene glycol dimethyl ether (POMDMEₙ₌₃) or a mixture comprising trioxymethylene glycol dimethyl ether is converted in the presence of an acidic catalyst. The acidic catalyst used may be a homogeneous or heterogeneous acidic catalyst. In general, the reaction is performed in the presence of small amounts of water. Suitable acidic catalysts are generally acids having a pKa of <4, mineral acids such as phosphoric acid, sulfuric acid, sulfonic acids such as trifluoromethanesulfonic acid and paratoluenesulfonic acid, heteropolyacids, acidic ion exchange resins, zeolites, aluminosilicates, silicon dioxide, aluminum oxide, titanium dioxide and zirconium dioxide. Oxidic catalysts may, in order to increase their acid strength, be doped with sulfate or phosphate groups, generally in amounts of from 0.05 to 10% by weight. The reaction can be performed in a stirred tank reactor (CSTR) or a tubular reactor. When a heterogeneous catalyst is used, a fixed bed reactor is preferred. In addition to trioxane, tetraoxane may also be formed in small amounts.

In step b), the product gas mixture a)—preferably in a first distillation column—is separated into a low boiler fraction b1 comprising trioxane, formaldehyde, water, methylene glycol, methanol, hemiformal (HFₙ₌₁), methylal and dioxymethylene glycol dimethyl ether (POMDMEₙ₌₂), and a high boiler fraction b2 comprising polyoxymethylene glycols (MG_{n>1}), hemiformals (HF_{n>1}) and polyoxymethylene glycol dimethyl ethers having 3 or more oxymethylene units (POMDME_{n>2}). Any tetraoxane formed is removed together with trioxane in the low boiler fraction b1, but may also be present to a certain degree in the high boiler fraction b2. The low boiler fraction b1 may additionally comprise, in small amounts, further secondary components such as formic acid and methyl formate.

The index n refers in each case to the number of oxymethylene units. Hemiformal refers to the formaldehyde/methanol hemiacetate. Hemiformals HF_{n>1} are the higher homologs of the formaldehyde hemiacetate with n CH₂O units.

The distillation columns used in the steps described below are columns of customary design. Useful columns include columns with random packing, tray columns and columns with structured packing; preference is given to tray columns and columns with structured packing. The term “low boiler fraction” is used for the mixture withdrawn in the upper part, the term “high boiler fraction” for the mixture withdrawn in the lower part of the column. In general, the low boiler fraction is withdrawn at the top of the column, the high boiler fraction at the bottom of the column. However, this is not obligatory. It is also possible to withdraw via side draws in the stripping or rectifying section of the column.

The first distillation column has generally from 1 to 50 plates, preferably from 3 to 30 plates. It is operated at a pressure of generally from 1 to 5 bar, preferably from 1 to 3 bar. The top temperature is generally from 0 to 150° C., preferably from 20 to 120° C.; the bottom temperature is generally from 70 to 220° C., preferably from 80 to 190° C.

The high boiler fraction b2 is preferably recycled into the reactor of step a).

The low boiler fraction b1 is subsequently—preferably in a second distillation column—separated into a low boiler fraction c1 comprising formaldehyde, water, methylene glycol, methanol, hemiformal (HFₙ₌₁), methylal and dioxymethylene glycol dimethyl ether (POMDMEₙ₌₂), and a high boiler fraction c2 comprising trioxane. Any tetraoxane present is removed together with the trioxane. The low boiler fraction c1 may additionally comprise, in small amounts, still further secondary components such as formic acid and methyl formate.

The second distillation column has generally from 1 to 50 plates, preferably from 3 to 30 plates. It is operated at a pressure of from 0.5 to 5 bar, preferably from 0.8 to 3 bar. The top temperature is generally from 0 to 140° C., preferably from 20 to 110° C.; the bottom temperature is generally from 80 to 220° C., preferably from 90 to 200° C.

In one variant of the process according to the invention, methanol and methyl formate are removed from the low boiler fraction c1. This can be done in a low boiler removal stage, in which case methylal and hemiformal are also removed as further low boilers. The low boiler fraction c1 is thus separated into a fraction d1 comprising water, methylene glycol, methanol, methyl formate and hemiformal (HFₙ₌₁), and a fraction d2 comprising formaldehyde, water and dioxymethylene glycol dimethyl ether (POMDMEₙ₌₂). The two fractions d1 and d2 may additionally also comprise formic acid. Fraction d2 is recycled into the trioxane synthesis reactor (step a)).

The trioxymethylene glycol dimethyl ether (POMDMEₙ₌₃) or the mixture comprising it can be obtained in a preceding synthesis by converting a mixture comprising formaldehyde and methanol and subsequently working-up the product mixture by distillation.

In one variant of the process according to the invention, the entire low boiler fraction c1 comprising formaldehyde, water, methylene glycol, methanol, hemiformal (HFₙ₌₁), methylal and dioxymethylene glycol dimethyl ether (POMDMEₙ₌₂) is recycled without further separation into the trioxymethylene glycol dimethyl ether synthesis.

In a further variant of the process according to the invention, the low boiler fraction c1, as described above, is separated into a low boiler fraction d1 comprising water, methylal, methylene glycol, methanol and hemiformal (HFₙ₌₁), and a high boiler fraction d2 comprising formaldehyde, water and dioxymethylene glycol dimethyl ether (POMDMEₙ₌₂), and fraction d1 is recycled into the trioxane synthesis reactor (step a)) and fraction d2 into the trioxymethylene dimethyl ether synthesis.

Alternatively, fraction d2 can also be discharged from the process as a by-product or be conducted into a formaldehyde synthesis preceding the POMDMEₙ₌₃ synthesis.

In a preferred embodiment, a mixture comprising tri- and tetraoxymethylene glycol dimethyl ether (POMDME_{n=3,4}) is used in the trioxane synthesis (step a)). It is preferably obtained by one of the processes described below.

Recently, polyoxymethylene dimethyl ethers have gained significance as diesel fuel additives. To reduce smoke and soot formation in the combustion of diesel fuel, polyoxymethylene dimethyl ethers are added to them as oxygen compounds which have very few C—C bonds, if any. In this context, POMDME_{n=3,4} have been found to be particularly effective. When mixtures comprising tri- and tetraoxymethylene glycol dimethyl ether (POMDME_{n=3,4}), though, are prepared in large amounts in order to find use as diesel fuel additives, a very economically viable process for trioxane preparation can be realized proceeding from these mixtures, since it would profit in this case from the economy of scale of the POMDME synthesis. In this case, a substream of the POMDME_{n=3,4} produced would thus be processed further to trioxane.

When the low boiler fraction c1 is separated into a fraction d1 comprising water, methylal, methylene glycol, methanol, methyl formate and hemiformal (HFₙ₌₁), and a fraction d2 comprising formaldehyde, water and dioxymethylene glycol dimethyl ether (POMDMEₙ₌₂), and the low boiler fraction d1 is recycled into the trioxane synthesis reactor (step a)), and when tri- and tetraoxymethylene glycol dimethyl ether (POMDME_{n=3,4}) are obtained by the process variants described below, the high boiler fraction d2 is preferably recycled into step A) of the synthesis variants described below.

In a first variant, a mixture of tri- and tetraoxymethylene glycol dimethyl ether (POMDME_{n=3,4}) is prepared by reacting formaldehyde with methanol and subsequently working-up the reaction mixture by distillation, comprising the steps of:
A) feeding aqueous formaldehyde solution and methanol into a reactor and converting them to a mixture A comprising formaldehyde, water, methylene glycol (MG), polyoxymethylene glycols (MG_{n>1}), methanol, hemiformals (HF), methylal (POMDMEₙ₌₁) and polyoxymethylene glycol dimethyl ethers (POMDME_{n>1});
B) feeding the reaction mixture A into a first distillation column and separating it into a low boiler fraction B1 comprising formaldehyde, water, methylene glycol, methanol, methylal and dioxymethylene glycol dimethyl ether (POMDMEₙ₌₂), and a high boiler fraction B2 comprising formaldehyde, water, methanol, polyoxymethylene glycols, hemiformals and polyoxymethylene glycol dimethyl ethers (POMDME_{n>1});
C) feeding the high boiler fraction B2 into a second distillation column and separating it into a low boiler fraction C1 comprising formaldehyde, water, methylene glycol, polyoxymethylene glycols, methanol, hemiformals, di-, tri- and tetraoxymethylene glycol dimethyl ether (POMDME_{n=2,3,4}), and a high boiler fraction C2 comprising polyoxymethylene glycols, high-boiling hemiformals (HF_{n>1}) and high-boiling polyoxymethylene glycol dimethyl ethers (POMDME_{n>4});
D) feeding the low boiler fraction C1 and if appropriate one or more recycle streams composed of formaldehyde, water, methylene glycol and polyoxymethylene glycols into a third distillation column and separating them into a low boiler fraction d1 comprising formaldehyde, water, methanol, polyoxymethylene glycols, hemiformals and dioxymethylene glycol dimethyl ether (POMDMEₙ₌₂), and a high boiler fraction D2 essentially consisting of formaldehyde, water, methylene glycol, polyoxymethylene glycols, tri- and tetraoxymethylene glycol dimethyl ether (POMDME_{n=3,4});
E) feeding the high boiler fraction D2 into a phase separation apparatus and separating it into an aqueous phase E1 essentially consisting of formaldehyde, water, methylene glycol and polyoxymethylene glycols, and an organic phase E2 comprising tri- and tetraoxymethylene glycol dimethyl ether (POMDME_{n=3,4});
F) feeding the organic phase E2 into a fourth distillation column and separating it into a low boiler fraction F1 essentially consisting of formaldehyde, water, methylene glycol and polyoxymethylene glycols, and a high boiler fraction F2 essentially consisting of tri- and tetraoxymethylene glycol dimethyl ether (POMDME_{n=3,4});
G) optionally feeding the aqueous phase E1 into a fifth distillation column and separating it into a low boiler fraction G1 essentially consisting of formaldehyde, water, methylene glycol and polyoxymethylene glycols, and a high boiler fraction essentially consisting of water.

In a step A), aqueous formaldehyde solution and methanol are fed into a reactor and converted to a mixture a comprising formaldehyde, water, methylene glycol, polyoxymethylene glycols, methanol, hemiformals, methylal and polyoxymethylene glycol dimethyl ether.

In step A), commercial aqueous formaldehyde solution can be used directly, or it can be concentrated beforehand, for example as described in EP-A 1 063 221. In general, the formaldehyde concentration of the aqueous formaldehyde solution is from 20 to 60% by weight. Methanol is preferably used in pure form. The presence of small amounts of other alcohols such as ethanol is not disruptive. It is possible to use methanol which comprises up to 30% by weight of ethanol.

Water, monomeric (free) formaldehyde, methylene glycol (MG) and oligomeric polyoxymethylene glycols of different chain length (MG_{n>1}) are present in aqueous solutions alongside one another in a thermodynamic equilibrium which is characterized by a particular distribution of the polyoxymethylene glycols of different length. The term “aqueous formaldehyde solution” also relates to formaldehyde solutions which comprise virtually no free water but rather essentially only water chemically bound in the form of methylene glycol or in the terminal OH groups of the polyoxymethylene glycols. This is the case especially for concentrated formaldehyde solutions. Polyoxymethylene glycols may, for example, have from two to nine oxymethylene units.

The acidic catalyst used may be a homogeneous or heterogeneous acidic catalyst. Suitable acidic catalysts are mineral acids, such as substantially anhydrous sulfuric acid, sulfonic acids such as trifluoromethanesulfonic acid and para-toluenesulfonic acid, heteropolyacids, acidic ion exchange resins, zeolites, aluminosilicates, silicon dioxide, aluminum oxide, titanium dioxide and zirconium dioxide. Oxidic catalysts may, in order to increase their acid strength, be doped with sulfate or phosphate groups, generally in amounts of from 0.05 to 10% by weight. The reaction can be performed in a stirred tank reactor (CSTR) or a tubular reactor. When a heterogeneous catalyst is used, a fixed bed reactor is preferred. When a fixed catalyst bed is used, the product mixture can subsequently be contacted with an anion exchange resin in order to obtain an essentially acid-free product mixture. In the less advantageous case, it is also possible to use a reactive distillation.

The reaction is effected generally at a temperature of from 0 to 200° C., preferably from 50 to 150° C., and a pressure of from 1 to 20 bar, preferably from 2 to 10 bar.

In a step B), the reaction mixture A is fed into a first distillation column and separated into a low boiler fraction B1 comprising formaldehyde, water, methylene glycol, methanol, methylal and dioxymethylene glycol dimethyl ether (POMDMEₙ₌₂), and a high boiler fraction B2 comprising formaldehyde, water, methanol, polyoxymethylene glycols, hemiformals and polyoxymethylene glycol dimethyl ethers (POMDME_{n>1}).

The first distillation column has generally from 3 to 50 plates, preferably from 5 to 20 plates. It is operated at a pressure of from 0.2 to 10 bar, preferably from 0.8 to 6 bar. The top temperature is generally from −20 to +160° C., preferably from +20 to 130° C.; the bottom temperature is generally from +30 to +320° C., preferably from +90 to +200° C.

In general, the low boiler fraction B1 is recycled into the POMDME reactor (step A)).

In a step C), the high boiler fraction B2 is fed into a second distillation column and separated into a low boiler fraction C1 comprising formaldehyde, water, methylene glycol, polyoxymethylene glycols, methanol, hemiformals, di-, tri- and tetraoxymethylene glycol dimethyl ether (POMDME_{n=2,3,4}), and a high boiler fraction C2 comprising polyoxymethylene glycols, high-boiling hemiformals (HF_{n>1}) and high-boiling polyoxymethylene glycol dimethyl ethers (POMDME_{n>4}).

The second distillation column has generally from 3 to 50 plates, preferably from 5 to 20 plates. It is operated at a pressure of from 0.1 to 10 bar, preferably from 0.2 to 6 bar. The top temperature is generally from +20 to +260° C., preferably from +20 to +230° C.; the bottom temperature is generally from +80 to +320° C., preferably from +100 to +250° C.

The high boiler fraction can be recycled into the POMDME reactor (step A)).

In one embodiment, the high boiler fraction C2 is fed together with methanol into a further (second) reactor and converted. This cleaves long-chain oligomeric polyoxymethylene glycols, hemiformals and polyoxymethylene glycol dimethyl ethers to shorter chains by reaction with methanol. It is possible to use the same acidic catalysts as in the first reactor. The reaction product is preferably fed into the (first) reactor (of step A)). The reaction product can also be fed directly into the first distillation column. The temperature in the second reactor is generally higher than in the first reactor and is generally from 50 to 320° C., preferably from 80 to 250° C. The second reactor is operated at a pressure of generally from 1 to 20 bar, preferably from 2 to 10 bar.

In a further step D), the low boiler fraction C1 and, if appropriate, one or more recycle streams composed of formaldehyde, water, methylene glycol and polyoxymethylene glycols are fed into a third distillation column and separated into a low boiler fraction D1 comprising formaldehyde, water, methanol, polyoxymethylene glycols, hemiformals and dioxymethylene glycol dimethyl ether (POMDMEₙ₌₂), and a high boiler fraction D2 essentially consisting of formaldehyde, water, methylene glycol, polyoxymethylene glycols, tri- and tetraoxymethylene glycol dimethyl ether (POMDME_{n=3,4}).

Here and hereinafter “essentially consisting of” means that the fraction in question consists of the components mentioned to an extent of at least 90% by weight, preferably to an extent of at least 95% by weight. The high boiler fraction D2 comprises in particular virtually no dioxymethylene glycol dimethyl ether any longer. Its content in the high boiler fraction D2 is generally <3% by weight.

The third distillation column has generally from 1 to 50 plates, preferably from 1 to 20 plates. It is operated at a pressure of from 0.1 to 10 bar, preferably from 0.2 to 6 bar. The top temperature is generally from 0 to +160° C., preferably from +20 to +130° C.; the bottom temperature is generally from +50 to +260° C., preferably from +80 to +220° C.

In general, the low boiler fraction D1 is recycled into the POMDME reactor (step A)).

In a step E), the high boiler fraction D2 is fed into a phase separation apparatus and separated into an aqueous phase E1 essentially consisting of formaldehyde, water, methylene glycol and polyoxymethylene glycols, and an organic phase E2 comprising tri- and tetraoxymethylene glycol dimethyl ether (POMDME_{n=3,4}). In addition, the organic phase E2 likewise also comprises formaldehyde, water, methylene glycol and polyoxymethylene glycols.

In a step F), the organic phase E2 is fed into a fourth distillation column and separated into a low boiler fraction F1 essentially consisting of formaldehyde, water, methylene glycol and polyoxymethylene glycols, and a high boiler fraction F2 essentially consisting of tri- and tetraoxymethylene glycol dimethyl ether (POMDME_{n=3,4}).

The fourth distillation column has generally from 1 to 100 plates, preferably from 1 to 50 plates. It is operated at a pressure of from 0.1 to 10 bar, preferably from 0.2 to 6 bar. The top temperature is generally from 0 to +160° C., preferably from +20 to +130° C.; the bottom temperature is generally from +100 to +260° C., preferably from +150 to +240° C.

The high boiler fraction F2 constitutes the product of value. It may comprise more than 99% by weight of POMDME_{n=3,4}.

In general, in a further (optional) step G), the aqueous phase E1 is worked up further. To this end, it is fed into a fifth distillation column and separated into a low boiler fraction G1 essentially consisting of formaldehyde, water, methylene glycol and polyoxymethylene glycols, and a high boiler fraction essentially consisting of water.

The fifth distillation column has generally from 1 to 30 plates, preferably from 1 to 20 plates. It is operated at a pressure of from 0.1 to 10 bar, preferably from 0.2 to 6 bar. The top temperature is generally from −20 to +120° C., preferably from +20 to +100° C.; the bottom temperature is generally from +40 to +180° C., preferably from +60 to +150° C.

The low boiler fractions F1 and/or G1 may be recycled as recycle streams into the third distillation column (step D)). They are preferably recycled into the third distillation column. The low boiler fractions F1 and/or G1 may, though, also be recycled as recycle streams into the POMDME reactor (step A)).

In a second, alternative process variant, a mixture of tri- and tetraoxymethylene glycol dimethyl ether (POMDME_{n=3,4}) is prepared by reacting formaldehyde with methanol and subsequently working-up the reaction mixture by distillation, comprising the steps of:
A) feeding aqueous formaldehyde solution and methanol into a reactor and converting them to a mixture A comprising formaldehyde, water, methylene glycol (MG), polyoxymethylene glycols (MG_{n>1}), methanol, hemiformals (HF), methylal (POMDMEₙ₌₁) and polyoxymethylene glycol dimethyl ethers (POMDME_{n>1});
B) feeding the reaction mixture A into a reactive evaporator and separating it into a low boiler fraction B1 comprising formaldehyde, water, methanol, methylene glycol, polyoxymethylene glycols, hemiformals, methylal and polyoxymethylene glycol dimethyl ethers (POMDME_{n>1}), and a high boiler fraction B2 comprising polyoxymethylene glycols, high-boiling hemiformals (HF_{n>1}) and high-boiling polyoxymethylene glycol dimethyl ethers (POMDME_{n>4}), and recycling the high boiler fraction B2 into the reactor (step A));
C) feeding the low boiler fraction B1 into a first distillation column and separating it into a low boiler fraction C1 comprising formaldehyde, water, methylene glycol, methanol, hemiformals, methylal, di-, tri- and tetraoxymethylene glycol dimethyl ether (POMDME_{n=2,3,4}), and a high boiler fraction C2 comprising polyoxymethylene glycols, high-boiling hemiformals (HF_{n>1}) and high-boiling polyoxymethylene glycol dimethyl ethers (POMDME_{n>4}), and recycling the high boiler fraction C2 into the reactive evaporator (step A));
D) feeding the low boiler fraction C1 into a second distillation column and separating it into a low boiler fraction D1 comprising formaldehyde, water, methanol, polyoxymethylene glycols, hemiformals, methylal and dioxymethylene glycol dimethyl ether (POMDMEₙ₌₂), and a high boiler fraction D2 substantially consisting of formaldehyde, water, methylene glycol, polyoxymethylene glycols, tri- and tetraoxymethylene glycol dimethyl ether (POMDME_{n=3,4});
E) feeding the high boiler fraction D2 into a phase separation apparatus and separating it into an aqueous phase E1 substantially consisting of formaldehyde, water, methylene glycol and polyoxymethylene glycols, and an organic phase E2 comprising tri- and tetraoxymethylene glycol dimethyl ether (POMDME_{n=3,4});
F) feeding the organic phase E2 into a third distillation column and separating it into a low boiler fraction F1 substantially consisting of formaldehyde, water, methylene glycol and polyoxymethylene glycols, and a high boiler fraction F2 substantially consisting of tri- and tetraoxymethylene glycol dimethyl ether (POMDME_{n=3,4});
G) optionally feeding the aqueous phase E1 into a fourth distillation column and separating it into a low boiler fraction G1 substantially consisting of formaldehyde, water, methylene glycol and polyoxymethylene glycols, and a high boiler fraction substantially consisting of water.

Deviating from the first variant, in step B), the reaction mixture A is fed into a reactive evaporator and separated into a low boiler fraction B1 comprising formaldehyde, water, methanol, methylene glycol, polyoxymethylene glycols, hemiformals, methylal and polyoxymethylene glycol dimethyl ether (POMDME_{n>1}), and a high boiler fraction B2 comprising polyoxymethylene glycols, hemiformals (HF_{n>1}) and polyoxymethylene glycols (POMDME_{n>3}). The high boiler fraction B2 is recycled into the reactor (step A)).

The reactive evaporator constitutes the bottom evaporator of the first distillation column. The fraction C2 returning from the first distillation column comprises polyoxymethylene glycols, high-boiling hemiformals (HF_{n>1}) and high-boiling polyoxymethylene glycols (POMDME_{n>4}). This fraction mixes in the reactive evaporator with the reaction mixture A which comprises a higher proportion of water, methanol, polyoxymethylene glycols, hemiformals and polyoxymethylene glycol dimethyl ethers of shorter chain length. Thus, in the reactive evaporator, this leads to cleavage of long-chain components to components of shorter chain length. The reactive evaporator is generally operated at the pressure of the first column. However, it can also be operated at higher pressure. The operating pressure of the reactive evaporator is generally from 0.1 to 20 bar, preferably from 0.2 to 10 bar; the operating temperature is generally from 50 to 320° C., preferably from 80 to 250° C.

In a step C), the low boiler fraction B1 is fed into a first distillation column and separated into a low boiler fraction C1 comprising formaldehyde, water, methylene glycol, methanol, hemiformals, methylal, di-, tri- and tetraoxymethylene glycol dimethyl ether (POMDME_{n=2,3,4}), and a high boiler fraction C2 comprising polyoxymethylene glycols, high-boiling hemiformals (HF_{n>1}) and high-boiling polyoxymethylene glycol dimethyl ethers (POMDME_{n>4}). The high boiler fraction C2 is returned to the reactive evaporator (step B).

The first distillation column generally has from 2 to 50 plates, preferably from 5 to 20 plates. It is operated at a pressure of from 0.1 to 10 bar, preferably from 0.2 to 6 bar. The top temperature is generally from 0 to 260° C., preferably from 20 to 230° C.; the bottom temperature is the temperature of the reactive evaporator.

In a step D), the low boiler fraction C1 is fed into a second distillation column and separated into a low boiler fraction D1 comprising formaldehyde, water, methanol, polyoxymethylene glycols, hemiformals, methylal and dioxymethylene glycol dimethyl ether (POMDMEₙ₌₂), and a high boiler fraction D2 substantially consisting of formaldehyde, water, methylene glycol, polyoxymethylene glycols, tri- and tetraoxymethylene glycol dimethyl ether (POMDME_{n=3,4}).

The second distillation column generally has from 1 to 50 plates, preferably from 1 to 20 plates. It is operated at a pressure of from 0.1 to 10 bar, preferably from 0.2 to 6 bar. The top temperature is generally from 0 to 160° C., preferably from 20 to 130° C.; the bottom temperature is generally from 50 to 260° C., preferably from 80 to 220° C.

In general, the low boiler fraction D1 is returned to the POMDME reactor (step A)).

In a step E), the high boiler fraction D2 is fed into a phase separation apparatus and separated into an aqueous phase E1 substantially consisting of formaldehyde, water, methylene glycol and polyoxymethylene glycols and an organic phase E2 comprising tri- and tetraoxymethylene glycol dimethyl ether (POMDME_{n=3,4}). The organic phase E2 additionally likewise comprises formaldehyde, water, methylene glycol and polyoxymethylene glycols.

In a step F), the organic phase E2 is fed into a third distillation column and separated into a low boiler fraction F1 substantially consisting of formaldehyde, water, methylene glycol and polyoxymethylene glycols, and a high boiler fraction F2 substantially consisting of tri- and tetraoxymethylene glycol dimethyl ether (POMDME_{n=3,4}).

The third distillation column generally has from 1 to 100 plates, preferably from 1 to 50 plates. It is operated at a pressure of from 0.1 to 10 bar, preferably from 0.2 to 6 bar. The top temperature is generally from 0 to +160° C., preferably from 20 to 130° C.; the bottom temperature is generally from +100 to +260° C., preferably from 150 to 240° C.

The high boiler fraction F2 constitutes the product of value. It may comprise more than 99% by weight of POMDME_{n=3,4}.

In general, in a further (optional) step G), the aqueous phase E1 is worked up further. To this end, it is fed into a fourth distillation column and separated into a low boiler fraction G1 substantially consisting of formaldehyde, water, methylene glycol and polyoxymethylene glycols, and a high boiler fraction substantially consisting of water.

The fourth distillation column generally has from 1 to 30 plates, preferably from 1 to 20 plates. It is operated at a pressure of from 0.1 to 10 bar, preferably from 0.2 to 6 bar. The top temperature is generally from −20 to +120° C., preferably from 20 to 100° C.; the bottom temperature is generally from +40 to +180° C., preferably from 60 to 150° C.

The low boiler fractions F1 and/or G1 may be returned as recycle streams to the second distillation column (step D)). They are preferably returned to the second distillation column.

The low boiler fractions F1 and/or G1 may also be returned as recycle streams to the POMDME reactor (step A)).

The invention will be illustrated in detail by the example which follows.

### EXAMPLE

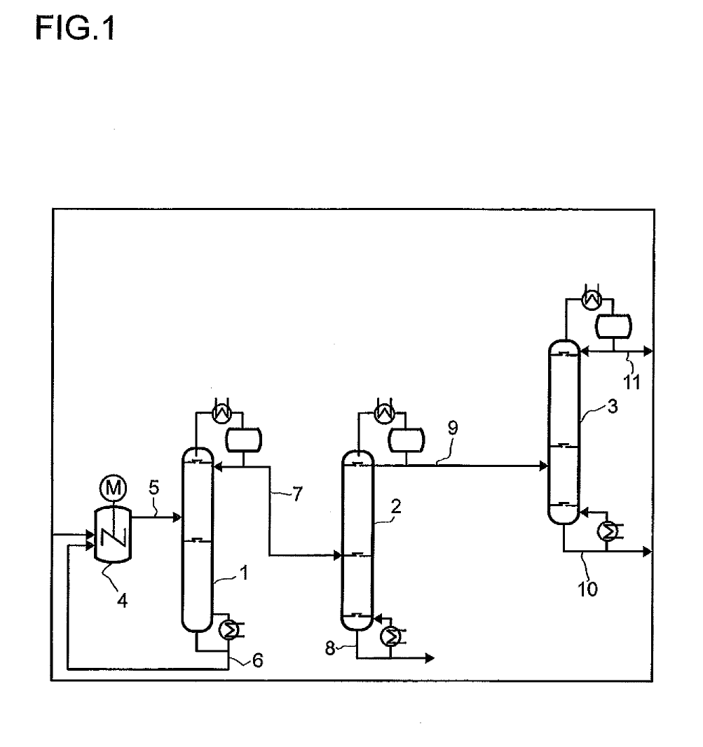

In the thermodynamic simulation of the process scheme shown in FIG. 1, the streams 6-11 listed in the table were obtained at the top or at the bottom of columns 1, 2 and 3.

The following parameters were selected: column 1 is operated at a pressure of 1.5 bar and 32 theoretical plates. The reflux ratio is 1.2, the top temperature is 73° C. and the bottom temperature 168° C. The feed 5 is to the 10^{th} tray of column 1. The bottom effluent 6 of column 1 is recycled into reactor 4.

The top effluent 7 of column 1 is fed to column 2 at the 12^{th} tray. Column 2 comprises 24 trays and is operated at a pressure of 2 bar. The top temperature is 75° C.; the bottom temperature is 140° C. The reflux ratio is 1.5.

The top effluent 9 of column 2 is fed to column 3. This stream is fed to the 20^{th} tray. The column has a total of 40 trays. It is operated at a pressure of 2.0 bar. The reflux ratio is 1.0. The top temperature is 63° C.; the bottom temperature is 83° C.

The composition of the individual streams is reported in the table below in % by weight.

| | Stream | | | | | | |
|---|---|---|---|---|---|---|---|
| | 5 | 6 | 7 | 8 | 9 | 10 | 11 |
| POMDMEn = 3 | 4% | 98% | 0% | 0% | 0% | 0% | 0% |
| Formaldehyde | 37% | 0% | 39% | 0% | 46% | 66% | 0% |
| Trioxane | 15% | 2% | 16% | 99% | 0% | 0% | 0% |
| POMDMEn = 2 | 17% | 0% | 18% | 1% | 21% | 30% | 0% |
| Methylal | 18% | 0% | 19% | 0% | 22% | 0% | 72% |
| Methanol | 6% | 0% | 6% | 0% | 7% | 0% | 24% |
| Methyl formate | 1% | 0% | 1% | 0% | 1% | 0% | 4% |
| Water | 2% | 0% | 2% | 0% | 3% | 4% | 0% |
| Amount [kg/h] | 100 | 4.1 | 95.9 | 14.9 | 81.0 | 56.0 | 25.0 |

1.-7. (canceled)

8. A process for preparing trioxane from trioxymethylene glycol dimethyl ether (POMDMEₙ₌₃) by converting trioxymethylene glycol dimethyl ether in the presence of an acidic catalyst and subsequent distillative workup of the reaction mixture, comprising the steps of:
a) feeding trioxymethylene glycol dimethyl ether (POMDMEₙ₌₃) or a mixture comprising trioxymethylene glycol dimethyl ether into a reactor and converting it in the presence of an acidic catalyst to a mixture a comprising trioxane, formaldehyde, water, methylene glycol (MG), polyoxymethylene glycols (MG_{n>1}), methanol, hemiformals (HF), methylal (POMDMEₙ₌₁) and polyoxymethylene glycol dimethyl ethers (POMDME_{n>1});
b) distillatively separating the reaction mixture a into a low boiler fraction b1 comprising trioxane, formaldehyde, water, methylene glycol, methanol, hemiformal (HFₙ₌₁), methylal and dioxymethylene glycol dimethyl ether (POMDMEₙ₋₂), and a high boiler fraction b2 comprising polyoxymethylene glycols (MG_{n>1}), hemiformals (HF_{n>1}) and polyoxymethylene glycol dimethyl ethers (POMDME_{n>2});
c) distillatively separating the low boiler fraction b1 into a low boiler fraction c1 comprising formaldehyde, water, methylene glycol, methanol, hemiformal (HFₙ₌₁), methylal and dioxymethylene glycol dimethyl ether (POMDMEₙ₌₂), and a high boiler fraction c2 comprising trioxane.

9. The process according to claim 8, wherein the high boiler fraction b2 is recycled into the reactor of step a).

10. The process according to claim 9, wherein the low boiler fraction c1 is separated into a low boiler fraction d1 comprising water, methylal, methylene glycol, methanol, methyl formate and hemiformal (HFₙ₌₁), and a high boiler fraction d2 comprising formaldehyde, water and dioxymethylene glycol dimethyl ether (POMDMEₙ₌₂), the high boiler fraction d2 being recycled into the reactor of step a).

11. The process according to claim 8, wherein the trioxymethylene glycol dimethyl ether (POMDMEₙ₌₃) or the mixture comprising it is obtained in a preceding synthesis by converting a mixture comprising formaldehyde and methanol and subsequently working-up the product mixture by distillation.

12. The process according to claim 11, wherein the low boiler fraction c1 comprising formaldehyde, water, methylene glycol, methanol, hemiformal (HFₙ₌₁), methylal and dioxymethylene glycol dimethyl ether (POMDMEₙ₌₂) is recycled into the trioxymethylene glycol dimethyl ether synthesis.

13. The process according to claim 10, wherein the low boiler fraction d1 is recycled into the trioxymethylene glycol dimethyl ether synthesis.

14. The process according to claim 8, wherein a mixture comprising tri- and tetraoxymethylene glycol dimethyl ether (POMDME_{n=3,4}) is used in step a).
